(19) United States
(12) Patent Application Publication
(10) Pub. No.: US 2022/0372072 A1
(43) Pub. Date: Nov. 24, 2022

(54) IMPROVED PURIFICATION PROCESSES FOR LIRAGLUTIDE

(71) Applicant: DR. REDDY'S LABORATORIES LIMITED, Hyderabad (IN)

(72) Inventors: Rajeev Rehani BUDHDEV, Hyderabad (IN); Nariyam Munaswamy SEKHAR, Hyderabad (IN); Karthik RAMASWAMY, Hyderabad (IN); Yagna Kiran Kumar KOMARAVOLU, Hyderabad (IN); Sunil Kumar GANDAVADI, Hyderabad (IN); Malleswara Reddy ANNARAPU, Hyderabad (IN); Peter MCCORMACK, Wirral (GB); Philip GAFFNEY, St Ives (GB); Sebastian KROLL, Hitchin (GB)

(21) Appl. No.: 17/762,246

(22) PCT Filed: Sep. 17, 2020
(86) PCT No.: PCT/IB2020/058678

§ 371 Date: Mar. 21, 2022

(30) Foreign Application Priority Data
Sep. 19, 2019 (IN) 201941037855
May 6, 2020 (IN) 202041019198

(51) Int. Cl. C07K 1/20; C07K 14/605; A61K 38/26; A61K 47/00

(52) U.S. Cl. CPC C07K 1/20; C07K 14/605; A61K 38/26; A61K 47/00

(57) ABSTRACT

The present application relates to improved and effective purification processes and also relates to method of increasing the solubility of for GLP-1 analog and its derivatives particularly Liraglutide. The purification process of present application is advantageous not only in terms of providing the highly pure peptide chemically but also in terms of affording peptide drug substance which is having good physical stability even at a large scale during holding or in-use period, while making drug substance compatible for formulation.

### INTRODUCTION

Aspects of the present application relates to improved and effective purification processes and also relates to method of increasing the solubility of for GLP-1 analog and its derivatives particularly Liraglutide.

Liraglutide, marketed under the brand name Victoza, is a long-acting glucagon like peptide agonist developed by Novo Nordisk for the treatment of type 2 diabetes.

Liraglutide is an injectable drug that reduces the level of sugar (glucose) in the blood. It is used for treating type 2 diabetes and is similar to exenatide (Byetta). Liraglutide belongs to a class of drugs called incretin mimetics because these drugs mimic the effects of incretins. Incretins, such as human-glucagon-like peptide-1 (GLP-1), are hormones that are produced and released into the blood by the intestine in response to food. GLP-1 increases the secretion of insulin from the pancreas, slows absorption of glucose from the gut, and reduces the action of glucagon. (Glucagon is a hormone that increases glucose production by the liver.) All three of these actions reduce levels of glucose in the blood. In addition, GLP-1 reduces appetite. Liraglutide is a synthetic (man-made) hormone that resembles and acts like GLP-1. In studies, Liraglutide treated patients achieved lower blood glucose levels and experienced weight loss.

Liraglutide, an analog of human GLP-1 acts as a GLP-1 receptor agonist. The peptide precursor of Liraglutide, produced by a process that includes expression of recombinant DNA in Saccharomyces cerevisiae, has been engineered to be 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. The molecular formula of Liraglutide is C₁₇₂H₂₆₅N₄₃ O₅₁ and the molecular weight is 3751.2 Daltons. It is represented by the structure of formula (I)

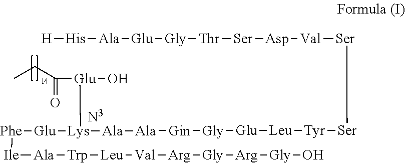

U.S. Pat. Nos. 7,572,884, 7,273,921, 6,451,974, 8,445,433, 9,260,474, 9,522,874 and International Application publication No. WO2013037266A1, WO2014199397A2, WO2016067271A1, WO2016046753A1, WO2017162650A1, WO2017127007A1, WO2017189925A1, WO2018033127A1, WO2017162650A1, WO2017138855A1, WO2018069295A1, WO2018104922A1, WO2019069274A1, WO2019143193A1, WO2019082138A1 reported various processes for Liraglutide and its intermediates.

Active ingredient of the approved product is being produced at an industrial scale by recombinant techniques. WO 1998/008871 describes reacting a recombinant expressed parent peptide with Na-hexadecanoyl-Glu(ONSu)-O^{t}Bu to obtain Liraglutide. It is desirable to provide methods for the large scale, full chemical synthesis of glucagon-like peptides such as Liraglutide. Chemical peptide synthesis has been extensively described in the literature. Two standard approaches to chemical peptide synthesis can be distinguished, namely liquid phase peptide synthesis (LPPS) and solid phase peptide synthesis (SPPS). Moreover, hybrid approaches can be utilized, where fragments are first synthesized by one of the above techniques and then joined together using the other. LPPS, also referred to as solution peptide synthesis, takes place in a homogenous reaction medium. Successive couplings yield the desired peptide. In SPPS, a peptide anchored by its C-terminus to an insoluble polymer resin is assembled by the successive addition of the protected amino acids constituting its sequence. Because the growing chain is bound to the insoluble support, the excess of reagents and soluble by-products can be removed by simple filtration. However, in particular for the synthesis of large peptides, resin-bound side products can accumulate in addition to side products formed during deprotection or due to degradation. As a result, the purification of the final product may very challenging.

Purification of glucagon-like peptides is particularly demanding due to their propensity to aggregate. It is known that glucagon and glucagon-like peptides tend to aggregate at acidic pH (e.g. European J. Biochem. 1 1 (1969) 37-42). The present invention provides methods for the production and purification of GLP-1 and GLP-1 analogs or its derivatives, in particular for the purification of Liraglutide.

Literature reported various purification methods like cation and anion-exchange purification process reported in U.S. Pat. No. 6,451,987B1, U.S. Pat. No. 6,444,788B1, ion-exchange chromatography in WO2005019261A1-, combination of ion-exchange and RP-HPLC by employing Tris-as a buffering agent or an additive and organic modifiers in loading solution in U.S. Pat. No. 8,710,181, counter-current purification system in U.S. Pat. No. 9,441,028, RP-HPLC under involving pH adjustment in a step-wise manner in U.S. Pat. No. 9,422,330, using metal ions in U.S. Pat. No. 9,447,163 and WO2003042249, simulated chromatographic separations using mathematical model in U.S. Pat. No. 9,766,217.

Additionally, WO2016046753A1 reported RP-HPLC purification using mobile phase comprising water and other mobile phase comprising acetonitrile and 01-4 alcohol. Further, multiple purifications involving use of basic buffer and organic solvent in 2^{nd} or 3rd chromatographic purifications are reported in WO2017162653A1, US20110313131A1, US20150051372A1.

The purification of liraglutide is difficult due to its long peptide chain and high hydrophobicity resulting from the presence of palmityl group. In the present invention, a purification method for liraglutide is provided obtained by solid phase chemical synthesis, which results in high purity and yield, and can be readily industrialized.

Other than purification of peptides, physical stability of therapeutic peptides plays an important role as they are required to have a shelf life of several years in order to be suitable for common use.

The instability of peptide compositions could be due to sensitivity towards chemical and physical degradation. Chemical degradation involves change of covalent bonds, such as oxidation, hydrolysis, racemization or crosslinking. Physical degradation involves conformational changes relative to the native structure of the peptide, i.e. secondary and tertiary structure, such as aggregation, precipitation or adsorption to surfaces. This property seems to encompass a transition from a predominant alpha-helix conformation to beta-sheets (Blundell T. L. (1983).

Thus, various excipients must often be added to pharmaceutical compositions of the glucagon-like peptides in order to improve their stability. The in-use period where the product may be transported and shaken daily at ambient temperature preferably should be several weeks. Thus, there is a need for pharmaceutical compositions of glucagon-like peptides which have improved stability.

Undissolved and/or insoluble GLP-1 peptide may be formed when GLP-1 solutions comprising water are agitated, exposed to hydrophobic surfaces or have large air/water interfaces. GLP-1 peptides are known to be prone to become undissolved and/or insoluble as a simple consequence of handling, for example during purification (e.g. Senderoff et al., Journal of Pharmaceutical Sciences, 1998, 87(2), 183-189). In addition, GLP-1 peptides may change into their undissolved and/or insoluble form during the process of their manufacturing. For example, mixing operations or continuous movement through a pump are common operations in large scale manufacturing processes and these operations cause the agitation, air/water interfaces and/or contact with hydrophobic surfaces that results in the undissolved and/or insoluble form of a GLP-1 peptide. The presence of the undissolved and/or insoluble form of GLP-1 peptides greatly affects large scale production of active GLP-1 peptides. In large scale production even small amounts of undissolved and/or insoluble GLP-1 peptide decrease cost efficiency of the production.

WO01/55213 allegedly describes using very alkaline pH in aqueous solution in order to dissolve insoluble GLP-1 peptide. WO2006/051110 allegedly describes using alkaline pH in aqueous solution in combination with certain heating conditions and incubation times in order to improve physical stability of the GLP-1 peptide, etc.

EP1396499A2, EP0747390B1, U.S. Pat. Nos. 7,632,806, 8,114,959, 8,748,376 describes various methods of stabilizing or reducing gelation like by adjusting the pH prior to lyophilisation on higher side i.e. greater than 8.1, or by subjecting the peptide solution to heat treatment etc.

While a number of aqueous formulations which stabilize peptide, polypeptide and protein compositions have been identified in the art, the destabilization of peptides, polypeptides and proteins in both formulation solutions and in solution during processing continues to create difficulty, especially in the up- and down-stream processing of these peptides.

Even though, the above mentioned prior art discloses diverse processes for purification of GLP-1 analog derivative i.e. Liraglutide, there is still a need for improved methods enabling the industrial production of highly pure glucagon-like peptide 1 analogs and derivatives for example in order to provide high yield methods for manufacture of active, soluble GLP-1 peptide as well as stable pharmaceutical products hereof, or in order to allow simpler production methods. Such improved methods involve transforming undissolved and/or insoluble GLP-1 peptide into active, soluble GLP-1 peptide.

Surprisingly, it has been found that the purification process of present application is advantageous not only in terms of providing the highly pure peptide chemically but also in terms of affording peptide drug substance which is having good physical stability even at a large scale during holding or in-use period, while making drug substance compatible for formulation.

The terms “glucagon-like peptide 1 analogs” and “GLP-1 analogs” are used herein interchangeably. The term “an analogue” is defined herein as a peptide wherein one or more amino acid residues of the parent peptide have been substituted by another amino acid residue. As used herein, they relate to peptides capable of binding to the GLP-1 receptor.

The GLP-1 analog derivatives of the present invention preferably have one or two Lys wherein the epsilon-amino group of one or both Lys is substituted with a lipophilic substituent. Liraglutide are preferred GLP-1 analog derivatives.

It will be understood by a person skilled in the art that a GLP-1 analogs or its derivatives as used herein may optionally bear any counter ions known in the art, depending on the purification process employed, such as anions or cations, such as e.g., chloride ions, acetate ions, carbonate ions, hydrocarbonate ions, sodium ions, potassium ions, any ions of a cleavage solution (e.g., TFA ions, bromide ions, perchlorate ions, ammonium ions) and/or cations or anions of residuals of protecting groups.

While the following teachings are often in respect to Liraglutide, it should be understood that they are likewise applicable to any other GLP-1 analogs or its derivatives.

### SUMMARY

In a first aspect of the present invention, there is provided a facile method of purifying GLP-1 analogs or its derivatives like Liraglutide which can achieve a high purity & physical stability suitable for use in pharmaceutical formulations. The process employs at least two or more of the following steps;
a) dissolving crude GLP-1 peptide analog or its derivative in suitable aqueous buffer,
b) Subjecting solution of step a) to first reversed phase HPLC purification, wherein a hydrocarbon bonded silica is used as a stationary phase, using mobile phase A, comprising aqueous basic buffer at a pH between about 8.0 and 8.5, and mobile phase B comprising acetonitrile, C₁-C₄alcohols, DMF, THF, acetone or their mixtures in desired ratio, and then eluting the desired peptide fractions;
c) Diluting the pooled desired peptide fractions obtained in step b) with water and subjecting to a second reversed phase HPLC purification, wherein a hydrocarbon bonded silica is used as a stationary phase, using mobile phase A′ comprising an aqueous mineral acid buffer optionally in combination with inorganic salts as additives at a pH below 3.0, and mobile phase B′ comprising acetonitrile, C₁-C₄alcohols, DMF, THF, acetone or their mixtures in desired ratio, and then eluting the desired peptide fractions; or
b′) Subjecting solution of step a) to first reversed phase HPLC purification wherein a hydrocarbon bonded silica is used as a stationary phase, using mobile phase A′ comprising an aqueous mineral acid buffer optionally in combination with mineral acid and/or inorganic salts as additives at a pH below 3.0, and mobile phase B′ comprising acetonitrile, C₁-C₄alcohols, DMF, THF, acetone or their mixtures in desired ratio, and then eluting the desired peptide fractions;
c′) Diluting the pooled desired peptide fractions obtained in step b) with basic buffer and subjecting to a second reversed phase HPLC purification, wherein a hydrocarbon bonded silica is used as a stationary phase, using mobile phase A, comprising Tris at a pH between about 8.0 and 8.5, and mobile phase B comprising acetonitrile, C₁-C₄alcohols, DMF, THF, acetone or their mixtures in desired ratio, and then eluting the desired peptide fractions;
and then,
d) Subjecting the pooled desired peptide containing fractions obtained in step c) or c′) for further purification, wherein a hydrocarbon bonded silica is used as a stationary phase, using a mobile phase A″ comprising an aqueous basic buffer at a pH between about 6.0-8.0, and mobile phase B″ comprising mobile phase B′ comprising acetonitrile, C1-C4 alcohols, DMF, THF, acetone or their mixtures in desired ratio, and then eluting the desired peptide fractions; and
e) Isolating the purified peptide from pooled peptide fractions obtained in step d) wherein the purified peptide fractions or purified peptide concentrate before drying has a pH of 6.0-7.5.

In a preferred embodiment, GLP-1 peptide analog or its derivative is Liraglutide

In a second aspect of the present invention, there is a provided a process for purification of a GLP-1 analogue or its derivatives thereof, on reverse phase high performance liquid chromatography (RP-HPLC) comprising a first and a second purification step with a mixture of aqueous buffer and an organic solvent for elution, characterized in that at least one chromatography purification is performed using an aqueous mobile phase comprising mineral acid buffer, optionally in combination with inorganic salts at a pH<3.0 and elution with an organic solvent.

In a third aspect of the present invention, there is a provided a gelation/fibrillation/aggregation resistant solution, comprising Liraglutide having 2.5-9.0% w/w of phosphate and 1.5-5.0% of sodium, relative to the total weight of dried material.

In a fourth aspect of the present invention, there is a provided a method for increasing the shelf-life of Liraglutide, the method comprising treating Liraglutide with an 1-6 mM aqueous basic phosphate buffer at pH 7.0-8.5.

The fifth aspect of the present invention provides Liraglutide of high purity at least >98% as obtained by process of present invention. In particular, the Liraglutide may contain less than 5%, less than 2%, less than 1%, less than 0.5%, less than 0.2%, or less than 0.1% by weight of any individual impurity as obtained by process of present invention.

The sixth aspect of the present invention provides pharmaceutical composition comprising GLP-1 analog or its derivative, particularly, Liraglutide obtainable according to any embodiment of the present invention, characterized in that said composition contains such peptide (particularly Liraglutide) at a purity above 99%, preferably above 99.5%, determined as a) the relative peak area observed in analytical RP-HPLC with UV detection at 220 nm, and b) shown lower high molecular weight impurities as observed in analytical size exclusion chromatography.

### DETAILED DESCRIPTION OF THE INVENTION

RP-HPLC techniques for purification of any protein or peptide, consisting of one or more equilibration steps, application or loading steps, wash steps, elution steps, and regeneration steps,

In a first aspect of the present invention, there is provided a facile method of purifying GLP-1 peptide analog or its derivative, particularly Liraglutide, which can achieve a high purity and physical stability product suitable for use in pharmaceutical formulations. The said process employs at least two of the following steps; a) dissolving crude GLP-1 peptide analog or its derivative in suitable aqueous buffer.

In a preferred embodiment, the suitable aqueous buffer is Tris buffer having pH between 8.0-8.5

In a preferred embodiment, GLP-1 peptide analog derivative is selected from Liraglutide. In a more preferred embodiment, it is Liraglutide.

Crude Liraglutide employed in step a) may be provided by any means known in the art as enunciated above. Exemplarily, it may be obtained from Solid Phase Peptide Synthesis (SPPS) or Liquid Phase Peptide Synthesis (LPPS) or a combination thereof. Alternatively, the plain polypeptide strand may also be obtained from a biotechnological method and the obtained polypeptide strand may be subsequently modified by chemical/synthetic means.

The term crude Liraglutide indicates the presence of “unwanted component” which is considered an impurity. Particularly preferred types of impurities are formed during synthesis and storage of Liraglutide and may exemplarily be selected from the group consisting of amino acids, peptides and derivatives thereof. In particular encompassed are impurities selected from the group consisting of amino acids, peptides, and derivatives thereof, which may result from processes such as premature chain termination during peptide synthesis, omission or unintended addition of at least one amino acid during peptide synthesis, incomplete removal of protecting groups, side reactions occurring during amino acid coupling or Fmoc deprotection steps, inter- or intramolecular condensation reactions, side reactions during peptide cleavage from a solid support, racemization, any other type of isomer formation, deamidation, (partial) hydrolysis, and aggregate formation. It is well known in the art that glucagon and glucagon- like peptides are prone to aggregate formation, and that low pH values often facilitate this process, i.e. that low pH values represent a destabilizing condition (cf., e.g., Wang et al., Mol. Pharm 12:41 1-419). Peptidic contaminations resulting from such processes as outlined above are sometimes referred to as “related substances”.

In a particularly preferred embodiment, the unwanted component is a peptidic impurity. As used herein, the expression “peptidic impurity” refers to unwanted peptidic compounds and comprises in particular derivatives of the peptide to be purified e.g. the result of oxidation or hydrolysis of amino acid side chains and/or a side product formed during peptide synthesis, truncated variants of the peptide to be purified that refers to continuous fragments, i.e. subsequences without gaps, of a given peptide, which lack one or more amino acids at the N-terminus and/or the C-terminus of the peptide sequence, deletion variants of the peptide to be purified refers to that refer to variants of the peptide to be purified, which differ from it in that their primary sequence lacks a single or multiple amino acid(s), and derivatives of such truncated and deletion variants.

In one embodiment, the unwanted component comprises covalent or non-covalent aggregates of the peptide to be purified. Such unwanted components are physiologically inactive or of unknown physiological effect. They are referred to herein as “high molecular weight (HMW) impurities”.

In a particularly preferred embodiment, the GLP-1 peptide analog derivative to be purified is Liraglutide. In a most preferred embodiment, the method according to the present invention allows to remove peptidic impurities so as to yield an essentially pure Liraglutide preparation. It was shown that the methods of the present invention yield essentially pure Liraglutide containing not more than 0.5% of any individual peptidic impurity, as assessed in terms of relative peak area observed by analytical chromatography, preferably with UV detection at a wavelength between 205 and 230 nm.

Chemical synthesis usually yields crude Liraglutide preparations having a purity of around 50 to 70%. It should however be understood that the crude Liraglutide of step a) may be characterized by any degree of purity below 100% (e.g. a purity above 40, 50, 60, 70, 80, or 90%) and that the present invention may also be advantageously applied to partially purified Liraglutide compositions.

In the context of the present invention, the term “purified” is used to designate peptide compositions which have been subjected to specific purification steps, e.g. to preparative chromatography. Such compositions may be highly or partially purified.

Unless noted otherwise, peptide purity is indicated herein as “HPLC purity”, i.e. as relative peak area observed in analytical reversed phase high performance liquid chromatography (RP-HPLC) with UV detection at a wavelength between 205 and 230 nm, i.e. at the absorption maximum of the peptide bond. In other words, the value is determined as % area of a given peak area divided by the sum of the areas of all observed peaks in a chromatogram obtained by analytical RP-HPLC with UV detection at a wavelength between 205 and 230 nm. This measure is common practice in the field, and the skilled person will routinely devise a product specific RP-HPLC protocol and perform the quantification according to the established guidelines set out in the United States Pharmacopeia. The suitability of the RP-HPLC protocol for the detection of peptidic contaminations is routinely assessed by determining the peak purity by LC-MS. Under the assumption that, due to their similar structure, all peptidic components have the same absorption, the RP-HPLC purity can be used as a proxy for a purity expressed as mass percentage [% (w/w)]. The skilled person is well aware of how to prepare samples for chromatographic purification.

For example, a dried crude Liraglutide preparation may be dissolved in aqueous buffers of a pH of 8.0-8.5. The sample concentration may be adjusted, inter alia, by drying, freeze-drying, partial evaporation of solvent, or ultrafiltration, and/or by dissolving or diluting the peptide preparation in a sample loading buffer, as the case may be.

b) Subjecting solution of step a) to a first reversed phase HPLC purification, wherein a hydrocarbon bonded silica is used as a stationary phase, using mobile phase A, comprising Tris at a pH between about 8.0 and 8.5, and mobile phase B comprising acetonitrile, C₁-C₄alcohols, DMF, THF, acetone or their mixtures in desired ratio, and then eluting the desired peptide fractions; Step b) involves the first RP-HPLC stage wherein mobile phases A and B are employed, preferably as a gradient elution. Mobile Phase A is an aqueous Tris buffer having pH between 7.5-8.5, preferably, between 8.0-8.5.

In a most preferred embodiment the peptide employed is Liraglutide.

As mentioned above, mobile phase B comprises acetonitrile, C₁-C₄alcohols, DMF, THF, acetone or their mixtures in desired ratio. In a preferred embodiment, acetonitrile and methanol are employed, preferably the ratio (vol:vol) of acetonitrile to the methanol in mobile phase B is from 60:40 to 95:5, more preferably 70:30 to 90:10, and most preferably 80:20.

According to preferred embodiments of the purification process of the present invention, step (b) is carried out by gradient elution, preferably from 75:25 v/v (mobile phase A: mobile phase B) to 35:65 v/v (mobile phase A: mobile phase B). c) Diluting the pooled desired peptide fractions obtained in step b) with water and subjecting to a second reversed phase HPLC purification, wherein a hydrocarbon bonded silica is used as a stationary phase, using mobile phase A′ comprising an aqueous mineral acid buffer, optionally in combination with inorganic salts as additives at a pH below 3.0, and mobile phase B′ comprising acetonitrile, C₁-C₄alcohols, DMF, THF, acetone or their mixtures in desired ratio, and then eluting the desired peptide fractions;

Step c) of the method involves dilution of the pooled desired peptide fractions from step b) and subjecting to a second reversed phase HPLC purification at a pH below 3.0.

Before & after loading the diluted desired pooled peptide fractions from step b) on to the column, the column is pre-equilibrated and after loading column is run with basic buffer having pH between 7.5-8.5, preferably, between 8.0-8.5. The basic buffer can be selected but not limited to Tris, sodium dihydrogen phosphate, disodium hydrogen phosphate, potassium dihydrogen phosphate, ammonium carbonate, ammonium hydroxide, sodium acetate, sodium carbonate, sodium bicarbonate, potassium carbonate, potassium acetate, or a combination thereof.

In a preferred embodiment, Tris-buffer having pH between 8.0-8.5 is employed.

As mentioned above, in a most preferred embodiment the peptide employed is Liraglutide.

The mobile phase A′ comprises mineral acid buffers selected but not limited from a group consisting of phosphoric acid, hydrochloric acid, nitric acid, perchloric acid, chloric acid, hydrofluoric acid, sulfuric acid and like.

The pH of mobile phase A′ is adjusted to below 3.0 using a base known to a person skilled in the art. It has been surprisingly found by the inventors that the base plays an important role in providing optimum resolution of the peptide of interest from the impurities. For example the pH of mineral acid can be adjusted from a group consisting of ammonia, sodium dihydrogen phosphate, disodium hydrogen phosphate, ammonium phosphate, ammonium carbonate, potassium carbonate, potassium acetate, sodium carbonate or a combination thereof. In a preferred embodiment the base is selected from a group consisting of ammonia and sodium dihydrogen phosphate. In yet another preferred embodiment, the base for adjusting the pH of mobile phase A′ is ammonia.

The mobile phase A′ comprises inorganic salts selected from a group consisting of NaCl, KCl, NH₄Cl, CaCl₂), sodium acetate, potassium acetate, ammonium acetate, sodium citrate, potassium citrate, ammonium citrate, sodium sulphate, potassium sulphate, ammonium sulphate, calcium acetate or mixtures thereof, most preferred are NaCl, NH₄Cl, KCl. or a combination thereof.

The mobile phase B′ comprises acetonitrile, C₁-C₄ alcohols, DMF, THF, acetone or their mixtures in desired ratio. Preferably acetonitrile and methanol are employed and the ratio (vol: vol) of acetonitrile to the methanol in mobile phase B′ is from 60:40 to 95:5, more preferably 70:30 to 90:10, and most preferably 80:20.

According to preferred embodiments of the purification process of the present invention, step (c) is carried out by gradient elution, preferably from 75:25 v/v (mobile phase A′: mobile phase B′) to 40:60 v/v (mobile phase A′:mobile phase B′) Alternatively, the steps b) and c) can be reversed and thus can be subjected to step b′) and step c′) according to below.
b′) Subjecting solution of step a) to first reversed phase HPLC purification wherein a hydrocarbon bonded silica is used as a stationary phase, using mobile phase A′ comprising an aqueous mineral acid buffer optionally in combination with inorganic salts as additives at a pH below 3.0, and mobile phase B′ comprising acetonitrile, C1-04 alcohols, DMF, THF, acetone or their mixtures in desired ratio, and then eluting the desired peptide fractions;
c′) Diluting the pooled desired peptide fractions obtained in step b) with water or basic buffer and subjecting to a second reversed phase HPLC purification, wherein a hydrocarbon bonded silica is used as a stationary phase, using mobile phase A, comprising Tris at a pH between about 8.0 and 8.5, and mobile phase B comprising acetonitrile, C₁-C₄ alcohols, DMF, THF, acetone or their mixtures in desired ratio, and then eluting the desired peptide fractions;

The aqueous mineral acid buffer, inorganic salts, and basic buffer can be selected from the list as mentioned above.
d) Diluting the pooled desired peptide fractions obtained in step c) with water or aqueous basic buffer and subjecting to further purification, wherein a hydrocarbon bonded silica is used as a stationary phase, using a mobile phase A″ comprising an aqueous basic buffer at a pH between about 6.0-8.0, and mobile phase B″ comprising mobile phase B′ comprising acetonitrile, C₁-C₄alcohols, DMF, THF, acetone or their mixtures in desired ratio, and then eluting the desired peptide fractions;

Step d) involves first dilution of the pooled desired peptide fractions obtained from step c) with water or aqueous basic buffer.

As mentioned above, in a most preferred embodiment the peptide employed is Liraglutide.

The mobile phase A″ comprises the aqueous basic buffer is selected from the group consisting of sodium dihydrogen phosphate, disodium hydrogen phosphate, sodium phosphate, potassium dihydrogen phosphate, dipotassium hydrogen phosphate, potassium phosphate, ammonium phosphate, ammonium carbonate, ammonium chloride, ammonium bicarbonate, ammonium sulphate, ammonium hydroxide, sodium acetate, sodium carbonate, sodium chloride, sodium bicarbonate, sodium phosphate and sodium sulphate, potassium carbonate, potassium acetate, or a combination thereof.

Preferably, the basic buffer is used at a concentration of 2 mM to 50 mM.

The pH of mobile phase A″ is preferably between 6.0 and 8.0.

The mobile phase B″ comprises polar organic solvents selected from acetonitrile, C₁-C₄alcohols, DMF, THF, acetone or mixtures thereof.

The elution can be done under isocratic or gradient mode or both, to improve the separation of impurities.
e) Isolating the purified peptide from pooled peptide fractions obtained in step d) wherein the purified peptide fractions or purified peptide concentrate before drying has a pH of 6.0-7.5.

As mentioned above, in a most preferred embodiment the peptide employed is Liraglutide.

It has been surprisingly found by the inventors that after elution the pooled fractions can be stabilized by addition of aqueous basic buffer which plays an important role in stabilizing the peptide of interest and increase the physical stability during holding time or in-use period and prevents uncontrolled precipitation. For example, dilution of the pooled desired peptide fractions with basic buffer selected from group consisting of sodium dihydrogen phosphate, disodium hydrogen phosphate, potassium dihydrogen phosphate, ammonium carbonate, ammonium hydroxide, sodium acetate, sodium carbonate, sodium bicarbonate, potassium carbonate, potassium acetate, or a combination thereof. In a preferred embodiment the dilution of the pooled desired peptide fractions obtained is performed with 1-6 mM phosphate buffer having pH between 7.5-8.5, preferably 8.0-8.5.

In yet another preferred embodiment, the process employs at least step a) and step c), or step a) and step b′) or step a) and step d).

For all the aforementioned aspects, following the final HPLC run, the Liraglutide fractions can be isolated by any suitable process, especially processes which enable a rapid removal of water at low temperature, such as by spray drying, or lyophilisation or precipitation at isoelectric point or by addition of suitable anti-solvent. Preferably, the drying step (e) comprises lyophilization.

This purified Liraglutide concentrate can be directly used to prepare a dried Liraglutide product which is suitable for preparing a pharmaceutical composition. Preferably, the concentrate employed in step (e) has a Liraglutide concentration of 2-80 mg/ml, more preferably 10-60 mg/ml, and most preferably 40-60 mg/ml.

In a preferred embodiment, the basic buffer is disodium hydrogen orthophosphate.

In yet another embodiment, the Liraglutide concentrate after lyophilization has anions up to 12% w/w and cations up to 9% w/w relative to the weight of Liraglutide. In a preferred embodiment, Liraglutide concentrate after lyophilization has anions of about 4% w/w and cations of about 4% w/w, relative to the weight of Liraglutide.

In yet another preferred embodiment, cations can be selected from sodium, potassium, ammonium, calcium, Tris and like, anion can be selected from phosphate, chloride, acetate, formate, carbonate, sulphate, bicarbonate, citrate, trifluoroacetate and like.

In yet another embodiment, the Liraglutide concentrate after lyophilization has phosphate of about 4% w/w and sodium of about 4% w/w relative to the weight of Liraglutide.

In yet another preferred embodiment, the GLP-1 analogue or its derivative thereof, prepared with a method for increasing its solubility and the shelf-life, the method comprising treating GLP-1 analogue or its derivative thereof with an aqueous basic buffer at pH 6.0-8.0 prior to isolation.

The above described purification process for Liraglutide is especially useful for purifying Liraglutide obtained by chemical peptide synthesis techniques. More preferably, the crude Liraglutide is obtained from a solid-phase or liquid phase peptide synthesis.

The second aspect of the present invention provided a process for purification of a GLP-1 analogue or its derivatives thereof, on reverse phase high performance liquid chromatography (RP-HPLC) comprising a first and a second purification step with a mixture of aqueous buffer and an organic solvent for elution, characterized in that at least one chromatography purification is performed using an aqueous mobile phase comprising acidic buffer, in combination with mineral acid and/or inorganic salts at a pH<3.0 and elution with an organic solvent.

The conditions of said purification are as explained above.

In a third aspect of the present invention, there is a provided a gelation/fibrillation/aggregation resistant solution, comprising Liraglutide having 2.5-9.0% w/w of phosphate and 1.5-5.0% of sodium, relative to the total weight of dried material.

In a fourth aspect of the present invention, there is a provided a method for increasing the shelf-life of Liraglutide, the method comprising treating Liraglutide with an 1-6 mM aqueous basic phosphate buffer at pH 7.0-8.5.

In a preferred embodiment, the above methods optionally further comprises, optionally an additional step of desalting the peptide followed by isolation of peptide by lyophilisation or precipitation at isoelectric point or by addition of suitable anti-solvent or combination thereof. Preferably, wherein desalting is performed by ion exchange chromatography, by size exclusion chromatography, or by ultrafiltration, RP-HPLC.

Size exclusion liquid chromatography is well known for analytical as well as preparative purposes in peptide chemistry. The method relies on the use of porous materials a stationary phase, where the pore size is selected such that only some components of a sample can enter into some of the pores. As a result, the accessible volume encountered by the various components varies, depending on each component's apparent molecular size. Hence, the components of the sample will elute from the column in the order of their apparent size, with large molecules eluting first. Ideally, the components of the sample do not interact with the surface of the stationary phase, such that differences in elution time result exclusively from differences in the solute volume each component can enter. Consequently, the composition of the mobile phase does not directly affect chromatographic resolution and can be adjusted with a view to sample properties or the needs of downstream processing steps.

It is envisaged to employ size exclusion chromatography after the RP-HPLC steps either for the separation of high molecular weight contaminants or for the removal of salt. Depending on the purpose, the skilled person will select a stationary phase with a suitable particle and pore size distribution. Preferred stationary phases for use with the present invention have pore sizes of 100-300 A (e.g. 100, 125, 145, 200 or 300 A) or molecular weight ranges of 0.7-10 kDa (e.g. <0.7, <1 0.5, 0.1-7, 1-5 or <10 kDa) or 1 0.5-30 kDa and particle sizes of 2-5 micrometer or 20-300 micrometer. Suitable commercial products comprise, e.g., Sephadex® G50 (GE Healthcare Life Sciences), Waters Acq uity™ BEH 200, Phenomenex Yarra™ SEC-2000, Tosoh Biosciences TSKgel® SuperSW2000, Sephadex® G-25 (GE Healthcare Life Sciences), Toyopearl® HW-40 (Tosoh Biosciences), Superdex® peptide (GE Healthcare Life Sciences) and Superdex®30 (GE Healthcare Life Sciences). Preferred mobile phases include ultra pure water, 10 mM aqueous sodium hydrogen phosphate at pH 7.5, or any buffer/solvent system compatible with the sample.

As used herein, the expressions “desalting” and “removal of salt” are used interchangeably for any method step which reduces a sample's salt content. For example, the salt content may be decreased by more than 50%, more than 60%, more than 70%, more than 80%, more than 90%, more than 95%, or more than 99%. In a preferred embodiment, the amount of buffer anions is reduced to levels below the detection level. Desalting may be performed by any suitable method. Besides size exclusion chromatography as described above, commonly used and well known options are dialysis, ion exchange chromatography and ultrafiltration. Ultrafiltration is a pressure-driven separation process, which relies on the use of a semipermeable membrane allowing for small buffer and solvent molecules to pass, but retaining the peptide of interest.

For the purpose of the present invention, it is preferred to use membranes having a molecular weight cut-off of not more than 3 kDa, e.g. 3 kDa, 2 kDa, 1 kDa, or below. The liquid passing through the membrane is referred to as “permeate” or “filtrate”, while the sample retained by the membrane is referred to as “retentate”. To avoid clogging of membrane pores, a tangential flow filtration format is advantageously employed. For the purpose of the present invention, it is preferred to use membranes compatible with organic solvents such as acetonitrile. In a particularly preferred embodiment, a polyethersulfone membrane with a molecular weight cut off of 1 kDa is used. It should however be understood that, as long as it provides a suitable molecular weight cut-off, the filter may be of any material known in the context of filtration, such as, e.g., plastic (e.g., nylon, polystyrene), metal, alloy, glass, ceramics, cellophane, cellulose, or composite material. The filter may be hydrophobic or hydrophilic. The surface of the filter may be neutral or positively charged or negatively charged.

The skilled person will routinely combine the methods of the present invention with suitable read-out techniques. For example, chromatographic steps may be monitored by following the UV absorbance of the eluate at a wavelength of 205-230 nm or 280 nm, and/or by following the eluate's conductivity. Moreover, chromatography may be combined with online or offline analysis by mass spectrometry, size exclusion UHPLC, ion exchange UHLPC, and/or reversed phase UHPLC, enzyme-linked immunosorbent assays (ELISA), and/or cell-based functional assays. In order to avoid deterioration of the peptide quality, the skilled person will carefully and routinely optimize the conditions of the purification steps including the sample storage. To this end, fractions may be, inter alia, pooled, precipitated, spray-died, freeze-dried, frozen, refrigerated, diluted, concentrated, and/or mixed with stabilizing buffers, bases, acids, or other substances. It is good practice to handle sensitive materials under stabilizing conditions. As a further example, it may be advantageous to freeze-dry Liraglutide preparations, preferably at a pH selected from a range of 6-7.5, preferably 7.0 to 7.5.

Reversed phase high performance liquid chromatography (RP-HPLC) employed above is well- known and widely used for peptide purification and analysis of peptide samples, i.e. for preparative as well as analytical purposes. The technique is based on hydrophobic association between the various components of a sample and a hydrophobic stationary phase, which association is disrupted by a solvent comprised in the mobile phase. Differential elution of the sample's components is generally achieved by gradually increasing the concentration of the solvent within the mobile phase.

From a practical perspective, this gradient is usually obtained by varying the proportions of a first and second elution buffer making up the mobile phase: The first mobile phase referred as Mobile Phase A or mobile phase A′ or mobile phase A″ comprises suitable aqueous buffer, while the mobile phase referred as Mobile Phase B or mobile phase B′ or mobile phase B″ comprises high amounts of the organic solvents. Hence, by increasing the proportion of mobile phase B/B′/B″, more hydrophobic components can be eluted from the stationary phase.

Elution is effected by gradually increasing the concentration of the acetonitrile/alcohol as a solvent. Without wishing to be bound by any theory, it is believed that the solvent competes with the association of the components to the stationary phase. In order to maintain a linear velocity, the skilled practitioner will adjust the flow rate of the mobile phase depending on the column diameter and taking account of the specifications of the equipment and stationary phase employed.

In one aspect of the invention an isocratic elution with respect to pH and/or the concentration of the mobile phase used in at least one elution step. When used herein the term “isocratic elution” when used with respect to pH or the concentration of the mobile phase” means elution under conditions in which pH respectively the concentration of the mobile phase in the elution composition remains constant throughout the procedure.

In once aspect of the invention the elution is performed in both gradient and isocratic manner. It has been surprisingly found by the inventors that simultaneous gradient and isocratic elution plays an important role in providing optimum resolution of the peptide of interest from the impurities.

As used herein, the term HPLC also includes ultra high performance liquid chromatography (UHPLC, also designated as UPLC). In one preferred embodiment, HPLC is UHPLC. More preferably, UHPLC is reversed phase UHPLC and may thus also be designated as RP-UHPLC. Therefore, in a particularly preferred embodiment, HPLC is RP-UHPLC. In the context of the present application, the expression “hydrocarbon bonded silica” refers to stationary chromatographic phases made from porous silica particles or silica gels having chemically bonded hydrocarbon moieties at their surface. It is understood that the type of chemical bond as well as the chemical nature of the bonded hydrocarbon moieties may vary. For example, a stationary phase for use with the present application may be made from porous silica particles having chemically bonded hydrocarbon moieties of 4 to 18, preferably 8 to 18, carbon atoms. Such hydrocarbon moieties are preferably linear alkyl chains. Preferred types of hydrocarbon bonded silica have hydrocarbon moieties with four (C4), six (C6), eight (C8), ten (010), twelve (C12), fourteen (C14), sixteen (C16), or eighteen (C18) carbon atoms. Particularly preferred types of hydrocarbon bonded silica have unbranched alkyl chains of four (C4), eight (C8), twelve (C12) or eighteen (C18) carbon atoms, i.e. butyl, octyl, dodecyl, or octadecyl moieties. C8 bonded silica, in particular n-octyl bonded silica, and/or C18 bonded silica, in particular n-octadecyl bonded silica, are even more preferred stationary phases for use in steps b), c), and optionally d) of a method according to the present invention. The stationary phase used in steps b) and c) and optionally d) may be the same or different in each of the steps. Preferably the stationary phase is the same. Particularly preferably, a single stationary phase (i.e., a single column) is used in steps b) and c) and optionally d). In the context of the present application, the expression “C8 bonded silica” is used to designate stationary chromatographic phases made from porous silica particles or silica gels having at their surface chemically bonded C8 hydrocarbon moieties, preferably linear octyl, i.e. n-octyl, moieties. Further, the expression “012 bonded silica” is used to designate stationary chromatographic phases made from porous silica particles or silica gels having at their surface chemically bonded C12 hydrocarbon moieties, preferably linear dodecyl, i.e. n-dodecyl, moieties. Likewise, the terms “018 bonded silica” or “ODS” are used herein interchangeably to refer to stationary chromatographic phases made from porous silica particles or silica gels having at their surface chemically bonded C18 hydrocarbon moieties, preferably linear octadecyl, i.e. n-octadecyl, moieties.

A wide range of hydrocarbon bonded silica materials is commercially available. Examples of stationary phases which can be used in present invention are Daisogel™ C18 ODS, Daiso ODS-Bio, Daiso-ODS-A-HG C18, Daisogel™ C8-Bio, YMC ODS-A, YMC Triart C8-L, Luna C8, Luna C18, Kromasil™ C18, and Kromasil™ C8 produced by Daiso, YMC, Phenomenex, and AkzoNobel, respectively.

The silica particles may be of 2 to 200 micrometer, preferably 2.5 to 20 micrometer, preferably 5-15 micrometer, and most preferably 10 micrometer, in diameter and may have a pore size of 50 to 1000 A, preferably of 80 to 400 A, preferably of 100 to 300 A, most preferably of (about) 100A.

In a preferred embodiment of the invention, all or parts of the chromatographic purification are carried out at a temperature selected from the range of 10−30° C., preferably 15-25° C. Likewise, all or parts of any of the optional further purification steps, i.e. size exclusion chromatography step (step e)) and/or an desalting step (step f)) may be carried out at a temperature selected from the range of 10−30° C., preferably 15-25° C.

The stationary phase used in steps a), b) and c), if present, is C4 or C8 or C18 bonded silica.

The fifth aspect of the present invention provides liraglutide of high purity as obtained by process of present invention. In particular, the Liraglutide may contain less than 5%, less than 2%, less than 1%, less than 0.5%, less than 0.2%, or less than 0.1% by weight of any individual impurity, as obtained by process of present invention.

The sixth aspect of the present invention provides a pharmaceutical composition comprising GLP-1 peptide analogs or its derivatives, preferably Liraglutide, obtainable according to any embodiment of the present invention, characterized in that said composition contains Liraglutide at a purity above 99%, preferably above 99.5%, determined as a) the relative peak area observed in analytical RP-HPLC with UV detection at 220 nm, and b) as the relative peak area observed in analytical size exclusion chromatography.

Another aspect of the present invention provides a pharmaceutical composition comprising GLP-1 peptide analogs or its derivatives, preferably Liraglutide, obtainable according to any embodiment of the present invention, and one or more pharmaceutically acceptable excipients selected from the group consisting of isotonicity agents, buffering agents, preservatives, pH adjusting agents, stabilizers, surfactants and chelating agents.

Pharmaceutically acceptable excipients, their concentrations and use in the pharmaceutical compositions is well-known to the skilled person. The concentration ranges of the excipients present in the pharmaceutical compositions are 1 mg/ml to 100 mg/ml of isotonicity agent, 0.1 mg/ml to 5 mg/ml of buffering agent, 0.1 mg/ml to 10 mg/ml of preservative, 0.1 mg/ml to 50 mg/ml of stabilizer and 0.1 mg/ml to 5 mg/ml of chelating agent.

The following Examples, including the experiments conducted and the results achieved, are provided for illustrative purposes only and are not to be construed as limiting the scope of the claims.

### Examples

### Example 1: Purification of Liraglutide

The crude Liraglutide of around 50-70% purity was dissolved in 100 mM Tris buffer (10 mg/ml) of pH: 8.0-8.5. The Reverse phase C18 (10 micron particle size) was equilibrated with 20 mM Tris buffer of pH: 8.0-8.5. The crude solution was loaded onto the C18, 10 microns silica and a purification cycle was performed with gradient of:

Mobile Phase A: 20 mM Tris buffer, pH=8.0-8.5

Mobile phase B: 8:2 Acetonitrile:methanol solution The desired fractions were collected whose purity >95% were pooled, diluted with water in 1:1 ratio and were then subjected to further purification using preparative HPLC on C4, 10 microns silica with the following gradient:

Mobile Phase A′: 20 mM Citric acid, pH=2-3 Mobile phase B′: 8:2 Acetonitrile:methanol solution The desired fractions were collected whose purity >98% were pooled, diluted with water in 1:1 ratio and were then subjected to further purification on C4, with the following eluants;

Mobile Phase A″: 4 mM aqueous disodium hydrogen phosphate dihydrate, pH=7.5-8.0 Mobile phase B″: 8:2 Acetonitrile:methanol solution The pooled fractions were subjected to distillation under vacuum to remove organic solvent, the pH of the solution in the end of the evaporation was 7.0-7.5. The solution was lyophilized to afford pure Liraglutide powder having >98.5%, individual impurity <0.5%.

### Example 2: Purification of Liraglutide

The crude Liraglutide of around 60% purity was dissolved in 100 mM Tris buffer (5 mg/ml) of pH-8.0-8.5. The reverse phase media C4 (10 micron particle size) was equilibrated with 0.1% TFA solution in water & prepared crude is loaded onto the column. The separation is performed with gradient elution with following mobile phases:

Mobile Phase A: 0.1% TFA in water, Mobile Phase B: 100% Acetonitrile The desired fractions were collected whose purity >90% were pooled, diluted with 20 mM Tris in water solution in 1:1.5 ratio and were then subjected to further purification using preparative HPLC on C18 (10 micron silica).

The second purification carried out with following mobile phases through gradient elution Mobile Phase A′: 20 mM Tris pH: 8.0-8.5

Mobile Phase B′: Acetonitrile:Methanol (8:2) The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A″: 4 mM Disodium hydrogen orthophosphate, pH: 7.5-8.5 Mobile phase B″: Acetonitrile: Methanol (8: 2)

The solution eluted with isocratic mode from the media whose purity >98.5% is further subjected to desolvatization through distillation process & the concentrated solution further lyophilized to afford pure Liraglutide whose purity >98.5% & other individual impurities <0.5%.

### Example 3: Purification of Liraglutide

The crude Liraglutide of around 60% purity was dissolved in 100 mM Tris buffer (5 mg/ml) of pH: 8.0-8.5. The reverse phase media C4 (10 micron) media was equilibrated with 0.2% orthophosphoric acid solution in water (pH:2.0-3.0) & prepared crude is loaded onto the column. The separation is performed with gradient elution with following mobile phases: Mobile Phase A: 0.2% orthophosphoric acid in water (pH: 2.0-3.0) Mobile Phase B: 100% Acetonitrile The desired fractions having purity >90% were pooled, diluted with 20 mM Tris in water solution in 1:1.5 ratio and were then subjected to further purification using preparative HPLC on C18, 10 micron silica.

The second purification carried out with following mobile phases through gradient elution Mobile Phase A′: 20 mM Tris pH: 8.0-8.5 Mobile Phase B′: Acetonitrile:Methanol (8:2).

The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A″: 4 mM Disodium hydrogen orthophosphate, pH: 7.5-8.5 Mobile phase B″: Acetonitrile: Methanol (8: 2).

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 4: Purification of Liraglutide

The crude liraglutide of around 60% purity was dissolved in 100 mM Tris buffer (5 mg/ml) of pH: 8.0-8.5. The reverse phase media C4 (10 micron) media was equilibrated with 20 mM citric acid solution in water (pH-2.0-3.0) & prepared crude is loaded onto the column. The separation is performed with gradient elution with following mobile phases: Mobile Phase A: 20 mM citric acid in water (pH: 2.0-3.0) Mobile Phase B: 100% Acetonitrile The desired fractions of purity >90% were pooled, diluted with 20 mM Tris in water solution in 1:1.5 ratio and were then subjected to further purification using preparative HPLC on C18 (10 micron silica).

The second purification carried out with following mobile phases through gradient elution Mobile Phase A′: 20 mM Tris pH: 8.0-8.5 Mobile Phase B′: Acetonitrile:Methanol (8: 2).

The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A″: 4 mM Disodium hydrogen orthophosphate, pH: 7.5-8.5

Mobile phase B″: Acetonitrile: Methanol (8: 2).

The solution eluted with isocratic mode from the media whose purity >98.5% is further subjected to desolvatization through distillation process & the concentrated solution further lyophilized to afford pure Liraglutide whose purity >98.5% & other individual impurities <0.5%.

### Example 5: Purification of Liraglutide

The crude liraglutide of — 60% purity was dissolved in 100 mM Tris buffer (5 mg/ml) of pH: 8.0-8.5. The reverse phase media C4 (10 micron) media was equilibrated with 20 mM citric acid solution in water (pH: 2.0-3.0) & prepared crude is loaded onto the column. The separation is performed with gradient elution with following mobile phases: Mobile Phase A: 20 mM citric acid in water (pH: 2.0-3.0) Mobile Phase B: 100% Acetonitrile The desired fractions of purity >90% were pooled, diluted with 10 mM Disodium hydrogen orthophosphate in water solution in 1:1.5 ratio and were then subjected to further purification using preparative HPLC on C18, 10 micron silica.

The second purification carried out with following mobile phases through gradient elution Mobile Phase A′: 10 mM Disodium hydrogen orthophosphate (pH: 8.0-8.5) Mobile Phase B′: Acetonitrile:Methanol (8:2). The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A″: 4 mM Disodium hydrogen orthophosphate, pH: 7.5-8.5 Mobile phase B″: Acetonitrile: Methanol (8: 2).

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 6: Purification of Liraglutide

The crude Liraglutide of around 60% purity was dissolved in 20 mM Tris buffer (5 mg/ml) with pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5 Mobile Phase B: Acetonitrile:Methanol (8:2) The desired fractions of purity >90% were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 20 mM Citric acid+150 mM Sodium chloride, pH: 2.0-3.0 Mobile Phase B′: Acetonitrile: Methanol (8: 2) The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A″: 4 mM Disodium hydrogen orthophosphate, pH: 7.5-8.5 Mobile phase B″: Acetonitrile: Methanol (8: 2).

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 7: Purification of Liraglutide

The crude liraglutide of around 60% purity was dissolved in 20 mM Tris buffer (5 mg/ml) of pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5 Mobile Phase B: Acetonitrile:Methanol (8:2) The desired fractions of purity >90% were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 0.1% orthophosphoric acid solution whose pH is adjusted to 2.0-3.00 with disodium hydrogen orthophosphate+20 mM Citric acid+150 mM Sodium chloride, pH: 2.0-3.0 Mobile Phase B′: Acetonitrile:Methanol (8:2) The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A: 4 mM Disodium hydrogen orthophosphate, pH: 7.5-8.5 Mobile phase B: Acetonitrile:Methanol (8: 2).

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 8: Purification of Liraglutide

The crude liraglutide of around 60% purity was dissolved in 20 mM Tris buffer (5 mg/ml) with pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5 Mobile Phase B: Acetonitrile:Methanol (8:2) The desired fractions were collected whose purity >90% were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 0.1% Orthophosphoric acid solution whose pH is adjusted to 2.0-3.00 with disodium hydrogen orthophosphate+150 mM Sodium chloride, pH: 2.0-3.0 Mobile Phase B′: Acetonitrile:Methanol (8:2) The desired fraction whose purity >98.0% were pooled together and further subjected to final purification with following mobile phase in C4 reverse phase media.

The fractions obtained from second purification were diluted with water & loaded to the C4 reverse phase media. The mobile phases for the final purification are as follows: Mobile phase A″: 4 mM Disodium hydrogen orthophosphate, pH: 7.5-8.5 Mobile phase B″: Acetonitrile:Methanol (8:2)

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 9: Purification of Liraglutide

The crude liraglutide of — 60% purity was dissolved in 20 mM Tris buffer (5 mg/ml) of pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5

Mobile Phase B: Acetonitrile:Methanol (8:2) The desired fractions of >90% purity were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 0.1% Orthophosphoric acid solution whose pH is adjusted to 2.0-3.0 with disodium hydrogen orthophosphate+20 mM Citric acid+150 mM Sodium chloride, pH: 2.0-3.0 Mobile Phase B′: Acetonitrile:Methanol (8: 2) The desired fraction whose purity >98.0% were pooled together and further subjected to final purification with following mobile phase in C4 reverse phase media.

The fractions obtained from second purification were diluted with water & loaded to the C4 reverse phase media. After loading the pooled fractions to the column, the media is washed with purified water for 3-5 column volumes. The mobile phases for the final elution are as follows: Mobile phase A″: Purified water Mobile phase B″: 100% Ethanol

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 10: Purification of Liraglutide

The crude liraglutide of — 60% purity was dissolved in 20 mM Tris buffer (5 mg/ml) with pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5

Mobile Phase B: Acetonitrile:Methanol (8:2) The desired fractions were collected whose purity >90% were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 0.1% orthophosphoric acid solution whose pH is adjusted to 2.0-3.00 with disodium hydrogen orthophosphate+20 mM Citric acid+150 mM Sodium chloride, Final pH: 2.0-3.0 Mobile Phase B′: Acetonitrile:Methanol (8: 2) The desired fraction whose purity >98.0% were pooled together and further subjected to final purification with following mobile phase in C4 reverse phase media.

The fractions obtained from second purification were diluted with water & loaded to the C4 reverse phase media. After loading the pooled fractions to the column, the media is washed with purified water for 3-5 column volumes. The mobile phases for the final elution are as follows: Mobile phase A″: 20 mM Tris in water, pH: 8.0-8.5 Mobile phase B″: Acetonitirile+Methanol (8:2)

The solution eluted with isocratic mode from the media whose purity >98.5% is further subjected to desolvatization through distillation process & the concentrated solution further lyophilized to afford pure liraglutide whose purity >98.5% & other individual impurities <0.5%.

### Example 11: Purification of Liraglutide

The crude liraglutide of ˜60% purity was dissolved in 20 mM Tris buffer (5 mg/ml) of pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5

Mobile Phase B: Acetonitrile:Methanol (8:2) The desired fractions were collected whose purity >90% were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 0.1% orthophosphoric acid solution whose pH is adjusted to 2.0-3.00 with disodium hydrogen orthophosphate+20 mM Citric acid+150 mM Sodium chloride, Final pH: 2.0-3.0 Mobile Phase B′: Acetonitrile:Methanol (8: 2) The desired fraction of purity >98.0% were pooled together and further subjected to final purification with following mobile phase in C4 reverse phase media.

The fractions obtained from second purification were diluted with water & loaded to the C4 reverse phase media. After loading the pooled fractions to the column, the media is washed with purified water for 3-5 column volumes. The mobile phases for the final elution are as follows: Mobile phase A″: 2 mM NaOH in water, pH: 8.0-9.5 Mobile phase B″: Ethanol

The solution eluted with isocratic mode from the media whose purity >98.5% is further subjected to desolvatization through distillation process & the concentrated solution further lyophilized to afford pure Liraglutide whose purity >98.5% & other individual impurities <0.5%.

### Example 12: Purification of Liraglutide

The crude Liraglutide of — 60% purity was dissolved in 20 mM Tris buffer (5 mg/ml) of pH 8.0-8.5. The reverse phase media C18 (10 micron) media was equilibrated with Mobile Phase A and Mobile phase B (95:5) Phase B and the prepared crude solution is loaded onto the column. The separation is performed with gradient elution with following mobile phases: Mobile Phase A: 20 mM Tris in water, pH: 8.0-8.5

Mobile Phase B: Acetonitrile:Methanol (8:2) After completion of elution, the desired fractions whose purity were >90% collected and pooled, then diluted with water solution and was then subjected to further purification using reverse phase media C18 (10 micron) media which was equilibrated with 20 mM aqueous Tris, pH=8.0-8.5 and Mobile phase B.

The second purification carried out with following mobile phases through gradient elution with following mobile phases: Mobile Phase A′: 0.3% orthophosphoric acid solution, pH 2.0-3.0 (pH adjusted with ammonia)+150 mM Sodium chloride, final pH: 2.0-3.0 Mobile Phase B′: Acetonitrile:Methanol (8: 2) After loading, the column was first washed with 20 mM aqueous Tris, pH=8.0-8.5 and Mobile phase B′ for initial 2-3 column volumes followed by gradient elution. The desired fraction whose purity >98.0% were pooled together, diluted with 20-50 mM Tris solution, pH=8.0-8.5 and then further subjected to final purification.

The above diluted fractions was then loaded on reverse phase media C18 (10 micron) media which was pre-equilibrated with mobile phase A″ and mobile phase B″ (05%:95%). The column was washed with water for 5-7 column volumes followed by elution through isocratic and gradient elution with following mobile phases: Mobile phase A″: 4 mM Disodium hydrogen orthophosphate (pH adjustment with orthophosphoric acid), pH: 8.0-8.5 Mobile phase B″: Acetonitrile:Methanol (8:2)

After completion of elution, the column was washed with 20 mM Tris in water for 2-3 column volumes followed by a wash with 20 mM Tris in water and acetonitrile: methanol in the ratio of (20:80) v/v for 2-3 column volumes.

The desired fraction whose purity >98.5% was further subjected to desolvatization through distillation process at/below 25° C. followed by filtration & washing with water and then lyophilized to afford Liraglutide.

### Example 13: Purification of Liraglutide

The crude Liraglutide of — 60% purity was dissolved in 20 mM Tris buffer (5 mg/ml) of pH: 8.0-8.5. The reverse phase media C18 (10 micron) media was equilibrated with 20 mM Tris in water & the prepared crude is loaded onto the column. The separation is performed with gradient elution with following mobile phases: Mobile Phase A: 20 mM Tris in water, pH: 8.0-8.5 Mobile Phase B: Acetonitrile:Methanol (8:2) The desired fractions of purity >90% were pooled, diluted with 20 mM Tris in water solution in 1:2 ratio and were then subjected to further purification using preparative HPLC on C4, 10 micron silica.

The second purification carried out with following mobile phases through gradient elution Mobile Phase A′: 0.1% Orthophosphoric acid solution whose pH is adjusted to 2.0-3.00 with disodium hydrogen orthophosphate+20 mM Citric acid+150 mM Sodium chloride, Final pH: 2.0-3.0 Mobile Phase B′: Acetonitrile:Methanol (8:2) The desired fraction whose purity >98.0% were pooled together and further subjected to final purification with following mobile phase in C4 reverse phase media.

The fractions obtained from second purification were diluted with water & loaded to the C4 reverse phase media. After loading the pooled fractions to the column, the media is washed with purified water for 3-5 column volumes. The mobile phases for the final elution are as follows: Mobile phase A″: 0.2% Triethylamine in water, pH: 7.5-9.0 Mobile phase B″: Ethanol

The solution eluted with isocratic mode from the media whose purity >98.5% is further subjected to desolvatization through distillation process & the concentrated solution further lyophilized to afford pure Liraglutide whose purity >98.5% & other individual impurities <0.5%.

### Example 14: Final Purification of Liraglutide

The fractions obtained from second purification were loaded on reverse phase media C4 (10 micron) media which was pre-equilibrated with mobile phase A″.

Mobile phase A″: 6 mM Disodium hydrogen orthophosphate (pH adjustment with orthophosphoric acid), pH: 8.0-8.5 Mobile phase B″: Acetonitrile:Methanol (8:2)

After sample loading, the column was washed with water followed by mobile phase A″ and then product was eluted 80% mobile phase B″ and 20% mobile phase A″. The desired fraction whose purity >98.5% was further subjected to desolvatization through distillation process at/below 25° C. followed by filtration & washing with water and then lyophilized to afford Liraglutide having ˜5% phosphate content.

### Example 15: Final Purification of Liraglutide

The fractions obtained from second purification were loaded on reverse phase media C4 (10 micron) media which was pre-equilibrated with mobile phase A″: mobile phase B″ in the ratio of 95:5.

Mobile phase A″: 4 mM Disodium hydrogen orthophosphate (pH adjustment with orthophosphoric acid), pH: 8.0-8.5 Mobile phase B″: Acetonitrile:Methanol (8:2)

After sample loading, the column was washed with water followed by elution with gradient elution protocol given below:

| Time | Flow rate | Buffer-A | Buffer-B |
|---|---|---|---|
| (min) | (cm/hr) | (%) | (%) |
| 0 | 100-170 | 100 | 0 |
| 18 | 100-170 | 100 | 0 |
| 9 | 100-170 | 75 | 25 |
| 13 | 100-170 | 65 | 35 |
| 36 | 100-170 | 50 | 50 |

The desired fraction whose purity >98.5% was further subjected to desolvatization through distillation process at/below 25° C. followed by filtration & washing with water and then lyophilized to afford Liraglutide.

### Example 16: Purification of Liraglutide

The desired fractions of purity >90% obtained from gradient elution with mobile phases: Mobile Phase A: 20 mM Tris in water, pH: 8.0-8.5 and Mobile Phase B: Acetonitrile: Methanol (8:2) were pooled, diluted with 20 mM Tris in water solution in 1:2 ratio and were subjected to further purification.

Purification carried out with following mobile phases through gradient elution with following mobile phases: Mobile Phase A′: 0.3% orthophosphoric acid solution, pH 2.0-3.0 (pH adjusted with ammonia)+150 mM Sodium chloride, final pH: 2.0-3.0 Mobile Phase B′: Acetonitrile:Methanol (8:2) After loading, the column was first washed with 95% Mobile Phase A′ and 5% Mobile phase B′ for initial 2-3 column volumes followed by elution with gradient elution protocol given below:

| Time | Flow rate | Buffer-A | Buffer-B |
|---|---|---|---|
| (min) | (cm/hr) | (%) | (%) |
| 0 | 100-170 | 80 | 20 |
| 15 | 100-170 | 80 | 20 |
| 65 | 100-170 | 50 | 50 |
| 130 | 100-170 | 45 | 55 |
| 205 | 100-170 | 40 | 60 |
| 290 | 100-170 | 35 | 65 |

The desired fraction whose purity >98.0% were pooled together and then further subjected to final purification.

The above diluted fractions was then loaded on reverse phase media C18 (10 micron) media which was pre-equilibrated with mobile phase A″ and mobile phase B″ (05%:95%). The column was washed with water for 5-7 column volumes followed by elution through isocratic and gradient elution with following mobile phases: Mobile phase A″: 4 mM Disodium hydrogen orthophosphate (pH adjustment with orthophosphoric acid), pH: 8.0-8.5 Mobile phase B″: Acetonitrile:Methanol (8:2)

After completion of elution, the desired fraction whose purity >98.5% was further subjected to desolvatization through distillation process at/below 25° C. followed by filtration & washing with water and then lyophilized to afford Liraglutide having ˜2.14% phosphate content.

1. A process for purification of a Liraglutide on reverse phase high performance liquid chromatography (RP-HPLC) comprising:
a) a first purification step characterized in that chromatography purification is performed using an mobile phase comprising Tris buffer at a pH 7.5-8.5 and organic solvents;
b) a second purification step characterized in that chromatography purification is performed using a mobile phase comprising aqueous mineral acid buffer and optionally inorganic salts at a pH<3.0 and organic solvents; and
c) a third purification step characterized in that chromatography purification is performed using an mobile phase comprising aqueous basic buffer at a pH between about 7.5-8.5 and organic solvents.

2. A process for purification of a Liraglutide on reverse phase high performance liquid chromatography (RP-HPLC) comprising:
a) a first purification step characterized in that chromatography purification is performed using an mobile phase comprising Tris buffer at a pH 7.5-8.5 and organic solvents;
b) a second purification step characterized in that chromatography purification is performed using an mobile phase comprising aqueous mineral acid buffer and optionally inorganic salts at a pH<3.0 and organic solvents; and
c) a third purification step characterized in that chromatography purification is performed using an mobile phase comprising a aqueous phosphate buffer at a pH between about 7.5-8.5 and organic solvents.

3. The process according to claim 1,
wherein the aqueous mineral acid buffer is selected from orthophosphoric acid, TFA and inorganic salt is selected from sodium chloride, sodium sulphate, ammonium chloride, ammonium acetate, ammonium sulphate, ammonium formate, potassium chloride, potassium sulphate.

4. The process according to claim 1, wherein the aqueous mineral acid buffer optionally further comprises organic acid selected from the group citric acid, formic acid, trifluoroacetic acid, acetic acid.

5. The process according to claim 1, wherein the aqueous basic buffer is selected from sodium dihydrogen phosphate, disodium hydrogen phosphate, sodium phosphate, potassium dihydrogen phosphate, dipotassium hydrogen phosphate, potassium phosphate, ammonium phosphate, ammonium carbonate, ammonium chloride, ammonium bicarbonate, ammonium sulphate, ammonium hydroxide, sodium acetate, sodium carbonate, sodium chloride, sodium bicarbonate, sodium phosphate and sodium sulphate, potassium carbonate, potassium acetate, or a combination thereof.

6. The process of claim 1, where the stationary phase in steps a) and c) are selected from C18, C8 and in step b) are selected from C4, C18 and C8.

7. The process of claim 6 wherein the stationary phase in steps a) and c) are C18 and in step b) is C4.

8. A process for purification of Liraglutide on reverse phase high performance liquid chromatography (RP-HPLC) comprising purifications with a mixture of aqueous buffer and an organic solvent for elution, characterized in that at least one chromatography purification is performed using an aqueous mobile phase comprising mineral acidic buffer, optionally in combination with inorganic salts at a pH<3.0 and elution with an organic solvent.

9. The process according to claim 8, stationary phase is run with basic buffer before elution with mineral acid buffer.

10. The process according to claim 1, wherein the purified fractions comprising Liraglutide after elution has a pH of 6.5-8.0.

11. The process according to claim 6, further comprising isolation of GLP-1 analogue or its derivatives by processes selected from lyophilization, pl precipitation, by addition of suitable anti-solvent or combinations thereof.

12. A method for increasing the shelf-life of Liraglutide, the method comprising treating Liraglutide with 1-6 mM aqueous basic phosphate buffer at pH 7.0-8.5.

13. Liraglutide comprising anion between 2.5-9.0% w/w and cation between 1.5-5.0% w/w relative to the total weight of dried material.

14. Liraglutide having 2.5-9.0% w/w of phosphate and 1.5-5.0% of sodium, relative to the total weight of dried material.

15. Liraglutide according to claim 14, wherein anion is phosphate & cation is sodium.

16. Isolated Liraglutide comprising about 2.5-9.0% w/w of phosphate and 1.5-5.0% w/w of sodium relative to the total weight of dried material which before lyophilization in water has a pH between 6.5-8.0.

17. A pharmaceutical composition comprising Liraglutide prepared according to claim 1, and pharmaceutically acceptable excipients.

18. A pharmaceutical composition prepared by combining isolated Liraglutide comprising about 2.5-9.0% w/w of phosphate and 1.5-5.0% w/w of sodium relative to the total weight of dried material with a pharmaceutically acceptable carrier.

19. Liraglutide manufactured by a process comprising the steps of
a) purifying Liraglutide using the method according to claim 1 or 2 claim 1 and
b) isolating Liraglutide.

20. A pharmaceutical composition prepared by a process comprising the steps of
a) purifying Liraglutide using the method according to claim 1 or 2 claim 1,
b) drying said purified Liraglutide, and
c) admixing said dried Liraglutide with a pharmaceutically acceptable excipient.

21. The process according to claim 2, wherein the aqueous mineral acid buffer is selected from orthophosphoric acid, TFA and inorganic salt is selected from sodium chloride, sodium sulphate, ammonium chloride, ammonium acetate, ammonium sulphate, ammonium formate, potassium chloride, potassium sulphate.

22. The process according to claim 2, wherein the aqueous mineral acid buffer optionally further comprises organic acid selected from the group citric acid, formic acid, trifluoroacetic acid, acetic acid.

23. The process of claim 2, where the stationary phase in steps a) and c) are selected from C18, C8 and in step b) are selected from C4, C18 and C8.

24. The process of claim 23 wherein the stationary phase in steps a) and c) are C18 and in step b) is C4.

25. The process according to claim 2, wherein the purified fractions comprising Liraglutide after elution has a pH of 6.5-8.0.

26. A pharmaceutical composition comprising Liraglutide prepared according to claim 2, and pharmaceutically acceptable excipients.

27. Liraglutide manufactured by a process comprising the steps of
a) purifying Liraglutide using the method according to claim 2 and
b) isolating Liraglutide.

28. A pharmaceutical composition prepared by a process comprising the steps of
a) purifying Liraglutide using the method according to claim 2,
b) drying said purified Liraglutide, and
c) admixing said dried Liraglutide with a pharmaceutically acceptable excipient.
